(19) United States
(12) Patent Application Publication
(10) Pub. No.: US 2016/0317710 A1
(43) Pub. Date: Nov. 3, 2016

(54) GRAFT TENSIONING METHODS

(71) Applicant: THE CENTER FOR ORTHOPEDIC RESEARCH AND EDUCATION, INC., Phoenix, AZ (US)

(72) Inventors: WILLIAM CICCONE, II, Queen Creek, AZ (US); MARC CRAIG JACOFSKY, Phoenix, AZ (US)

(21) Appl. No.: 15/209,099

(22) Filed: Jul. 13, 2016

Related U.S. Application Data
(63) Continuation of application No. 12/784,701, filed on May 21, 2010, now abandoned.

(51) Int. Cl. A61L 27/36; A61F 2/08

(52) U.S. Cl. CPC A61L 27/3691; A61F 2/08; A61L 27/3662; A61L 2430/10; A61L 2430/40

(57) ABSTRACT

A graft tensioning device comprising a basin capable of containing a fluid, a heating element capable of heating the fluid to an appropriate temperature, a tray disposed within the basin, and a graft holding fixture for maintaining a tissue graft in tension, permanently or removably secured to the tray, wherein the graft holding fixture and tissue graft may be submersed within the fluid. The device may further comprise a base, a shelf and/or a cover. The device may be reusable, disposable or some combination thereof.

### CROSS-REFERENCE TO RELATED APPLICATION

This application is a continuation of U.S. application Ser. No. 12/784,701, filed May 21, 2010. The disclosure of the above-referenced application is hereby incorporated herein by reference in its entirety.

### FIELD

This application relates generally to medical devices, and specifically to medical devices used to prepare tissue grafts prior to and during joint stabilization procedures.

### BACKGROUND

Joint stabilization procedures, including soft tissue replacement surgeries such as reconstruction or replacement of the anterior cruciate ligament, are performed to restore stability to an injured joint. During such surgeries a tissue graft, such as a tendon graft or a bone-tendon-bone graft, is often used to replace failed tissues. Tissue grafts may be harvested from the patient (autograft), a cadaver (allograft), or even from a third-party living donor.

Tissue grafts are generally harvested or prepared early in the surgical procedure and then preconditioned prior to implantation or reimplantation. The harvested graft, often tendon strands, may be sutured together into a bundle in preparation for implantation. The graft is then typically preconditioned on a graft board, which involves holding the graft in tension to remove laxity from the tissue and, in the case of sutured grafts, to ensure proper alignment of the individual strands of tendon. It is understood that preconditioning the graft tends to minimize the influence of viscoelasticity after implantation.

Even when prepared under tension, it is common for grafts to continue to lengthen and stretch after implantation. This relaxation of the graft contributes to post-operative joint laxity resulting in instability of the joint.

Recent research, as published in articles such as Viscoelasticity and Temperature Variations Decrease Tension and Stiffness of Hamstring Tendon Grafts Following Anterior Cruciate Ligament Reconstruction, THE JOURNAL OF BONE AND JOINT SURGERY, 2006, p. 1071, Dr. William J. Ciccone II, Derek R Bratton, David M. Weinstein, and John J. Elias, has shown that the tension and the stiffness of a graft may significantly decrease due to both stress relaxation and an increase in temperature. This new information has led doctors and scientists to believe that the graft tension and stiffness achieved immediately following reconstruction is not maintained postoperatively due to the change in conditions between the operating room and the inside of the human body. Because postoperative joint laxity is now known to be at least partially a result of the transition between the temperature and atmospheric conditions of the operating room and the temperature and atmospheric conditions of the body, it has been determined that maintaining grafts in conditions similar to those of the body during and/or throughout the procedure may reduce or eliminate the influence of a post implantation temperature increase on graft properties.

In view of the foregoing, there is a need to provide a graft tensioning device that allows a graft to be maintained at conditions similar to those found within the human body during the preconditioning process. The present invention addresses one or more of these needs.

### SUMMARY

According to one embodiment, the device comprises a basin capable of containing a fluid, a heating element disposed within or near the basin and capable of heating the fluid to an appropriate temperature, a tray disposed within the basin, and a graft holding fixture disposed atop the tray for maintaining a graft in tension, wherein the graft holding fixture and graft may be submersed within the fluid.

According to another embodiment, the device further includes a base positioned in supportive relation to the basin, the heating element, the tray and the graft holding fixture.

In various embodiments, the device may include a shelf.

In various embodiments, the device may further include a cover.

In various embodiments, the device may further include a tube sizer.

In various embodiments, the device may be reusable, disposable, or partially disposable.

Other independent features and advantages of the graft tensioning device will become apparent from the following Detailed Description, taken in conjunction with the accompanying drawings which illustrate, by way of example, the principles of the invention.

### BRIEF DESCRIPTION OF DRAWINGS

FIG. 1 is an oblique plan view of a graft tensioning device, according to an embodiment of the invention;

FIG. 2 is a sectional view of the graft tensioning device, according to another embodiment of the invention;

FIG. 3 is an oblique plan view of the graft tensioning device, according to yet another embodiment of the invention;

FIG. 4 is a sectional view of the graft tensioning device, according to a further embodiment of the invention;

FIG. 5 is a sectional view of the graft tensioning device, according to another embodiment of the invention;

FIG. 6 is an oblique plan view of the graft tensioning device, according to a further embodiment of the invention;

FIG. 7 is an end view of the graft hook, according to an embodiment of the invention;

FIG. 8 is an oblique plan view of the graft tensioning device, according to another embodiment of the invention;

FIG. 9 is a sectional view of the graft tensioning device, according to a further embodiment of the invention;

FIG. 10 is a sectional view of the graft tensioning device, according to another embodiment of the invention;

FIG. 11 is a sectional view of the graft tensioning device according to yet another embodiment of the invention;

FIG. 12 is a sectional view of the graft tensioning device according to a further embodiment of the invention;

FIG. 13 is a plan view of the graft tensioning device according to another embodiment of the invention; and

FIG. 14 is a sectional view of the graft tensioning device according to the embodiment set forth in FIG. 13.

### DETAILED DESCRIPTION

The following Detailed Description of the invention is merely exemplary in nature and is not intended to limit the invention or the application and uses of the invention. Furthermore, there is no intention to be bound by any theory presented in the preceding Background or Summary or the following Detailed Description. Reference will now be made in detail to exemplary embodiments, examples of which are illustrated in the accompanying drawings. Wherever possible, the same reference numbers will be used throughout the drawings to refer to the same or like parts.

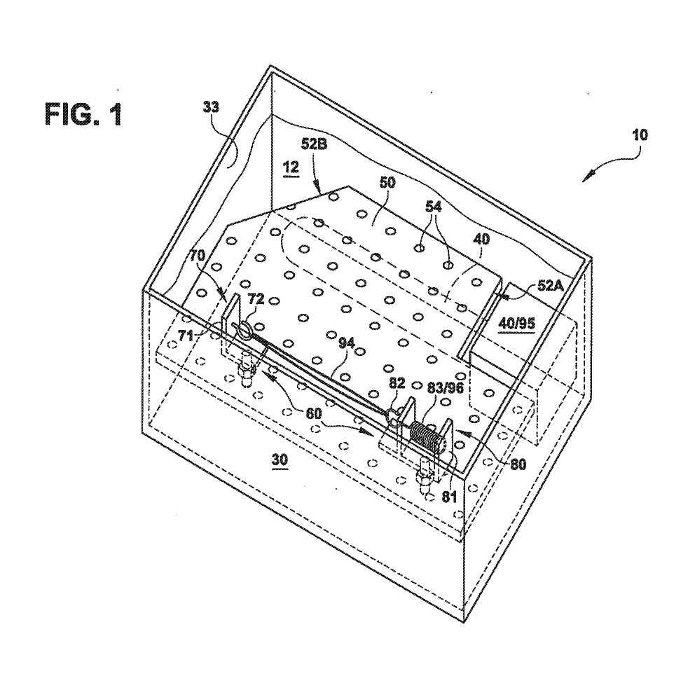

According to one embodiment, as illustrated in FIG. 1, the graft tensioning device 10 comprises a basin 30, a heating element 40 disposed within or near the basin 30, a graft holding fixture 60 and a tray 50 in supportive relation to the graft holding fixture 60. The graft tensioning device 10 is designed to maintain a graft 94 in tension prior to, during and/or after a surgical procedure. The graft 94 may be a soft tissue graft, such as a tendon strand, or a graft that includes non-soft tissue, such as a bone-tendon-bone graft. The graft 94 may consist of a single strand or multiple strands as is known in the art.

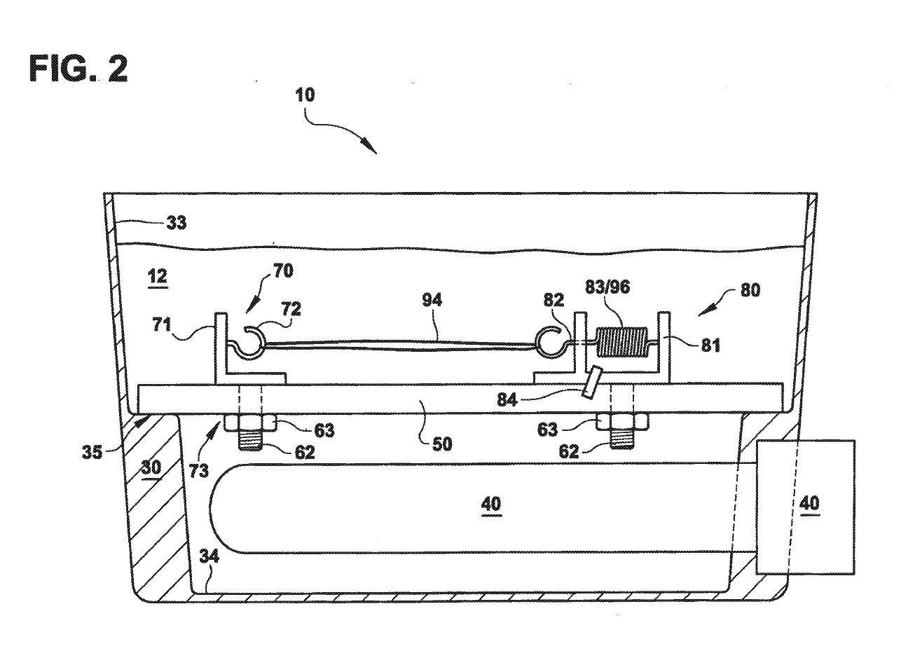

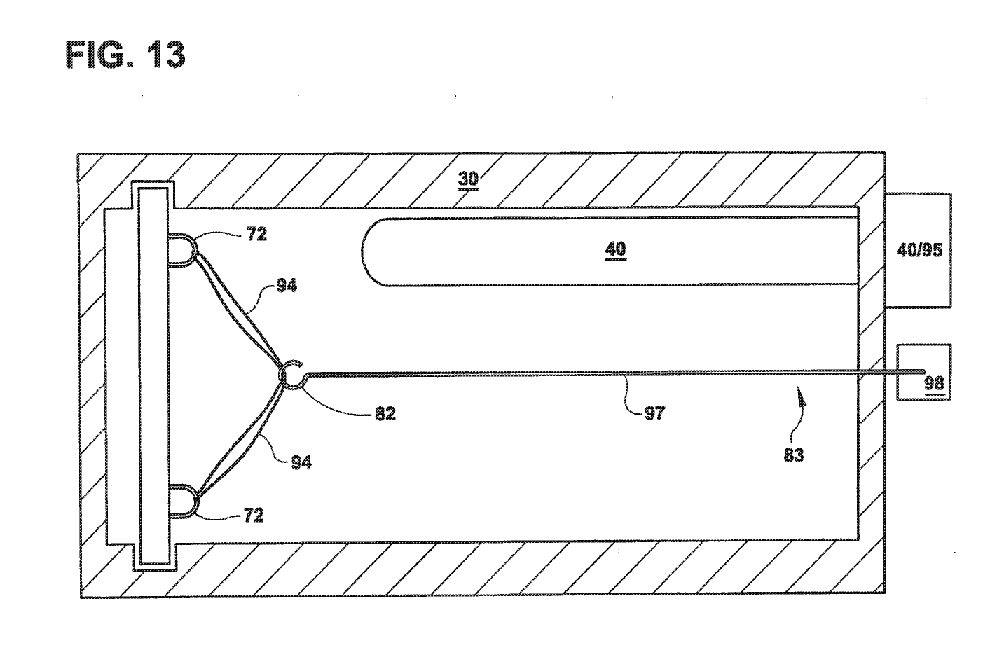

The basin 30 consists of one or more walls 33 and a floor 34. The basin 30 is capable of containing a fluid 12, including, but not limited to, a sterile saline or antibiotic rinse solution. The basin 30 may be any shape or configuration suitable for receiving the tray 50 and the fluid 12. According to the embodiment of FIGS. 1, 3, 6 and 8, the basin 30 is rectangular in shape, however any shape is within the inventive concept. As shown in FIG. 13, the one or more walls 33 of the basin 30 may consist of a shape necessary to account for placement of other components of the device. The basin 30 is preferably made of polyvinylchloride, HDPE, PETE, LDPE, polypropylene, or stainless steel, however any material is within the inventive concept. As shown in FIG. 2, the basin 30 may further comprise a ledge 35 designed to receive the perimeter of the tray 50 and support the tray 50 above the floor 34 of the basin 30.

The heating element 40 may be any element capable of heating the fluid 12 from approximately room temperature to approximately at or above body temperature in a time that is realistic in a surgical environment. For example, the heating element 40 may comprise electric power in watts appropriate to heat the fluid 12 from approximately room temperature to approximately at or above body temperature in a time that is realistic in a surgical environment. Alternatively, the heating element 40 may comprise chemical reactants capable of reacting to generate heat sufficient to heat the fluid 12 from approximately room temperature to approximately at or above body temperature in a time that is realistic in a surgical environment. The heating element 40 is preferably a submersible heating element, and may further comprise a temperature control device such as a thermocouple. As shown in FIGS. 1, 2, 8 and 9, the heating element 40 is disposed within the basin 30 such that the heating element 40 may be used to warm the fluid 12 to an appropriate temperature. Alternatively, the heating element 40 is disposed outside the basin 30 such that the heating element 40 is capable of warming the fluid 12 within the basin 30. According to one embodiment, the heating element 40 is capable of heating one liter of fluid to approximately 98 degrees in less than fifteen minutes. By way of example only, a 400 watt submersible heating element has been shown to heat approximately one liter of fluid from approximately 65 to approximately 99 degrees in approximately 12 minutes. The heating element 40 is further capable of maintaining a fluid at relatively consistent temperature. Consistency is maintained using a thermocouple, a microswitch or other temperature control device 95. The power source for the heating element 40 may be alternating current or direct current depending on the configuration and intended method of using the device 10. According to yet another embodiment, a preheated fluid 12 is used in lieu of heating element 40.

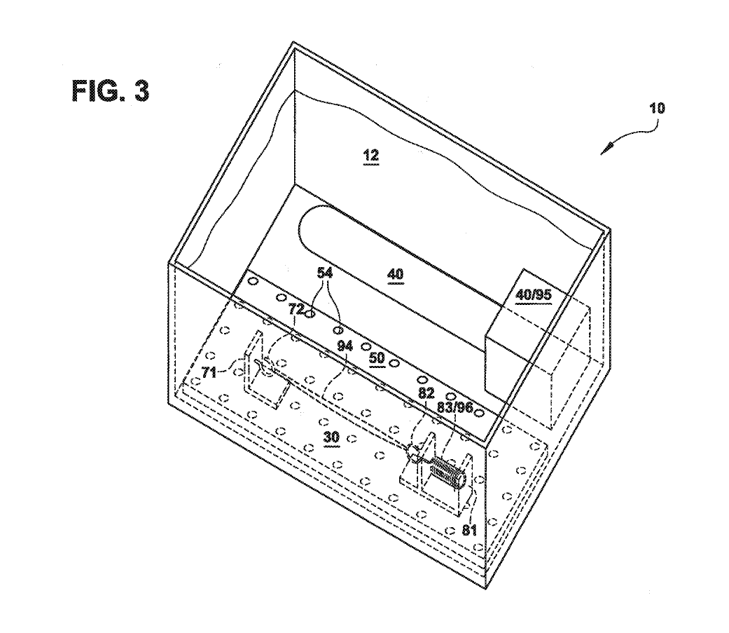

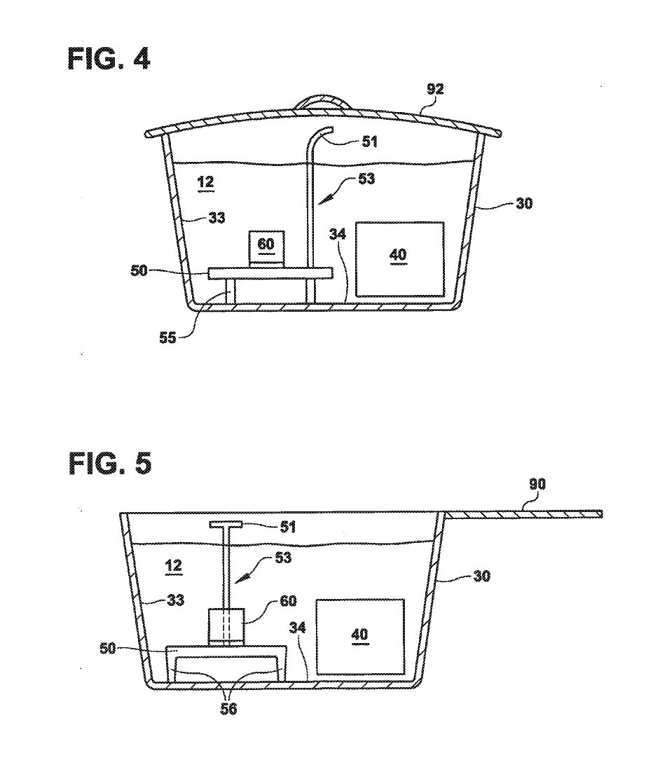

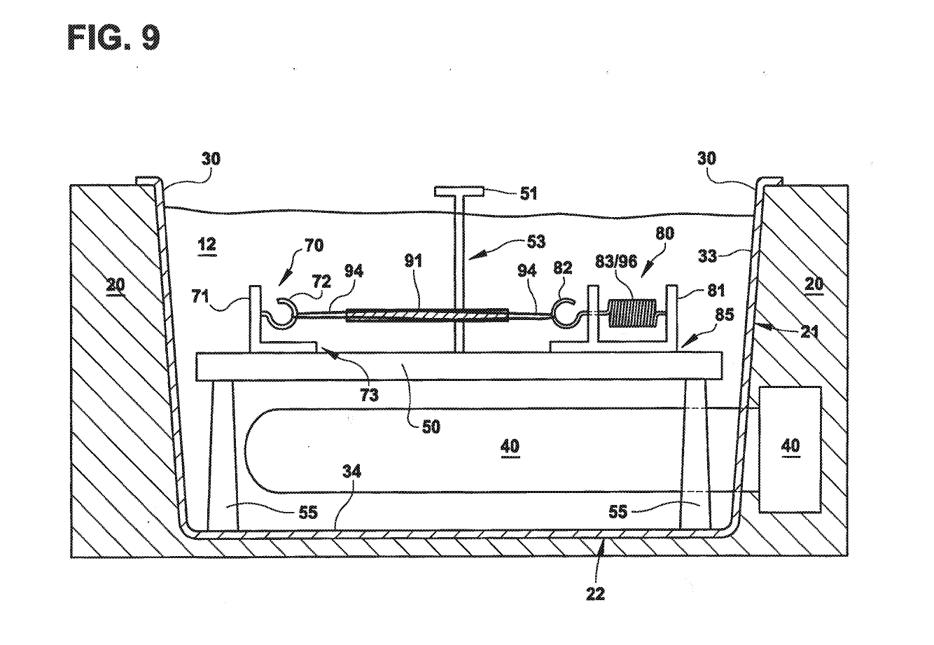

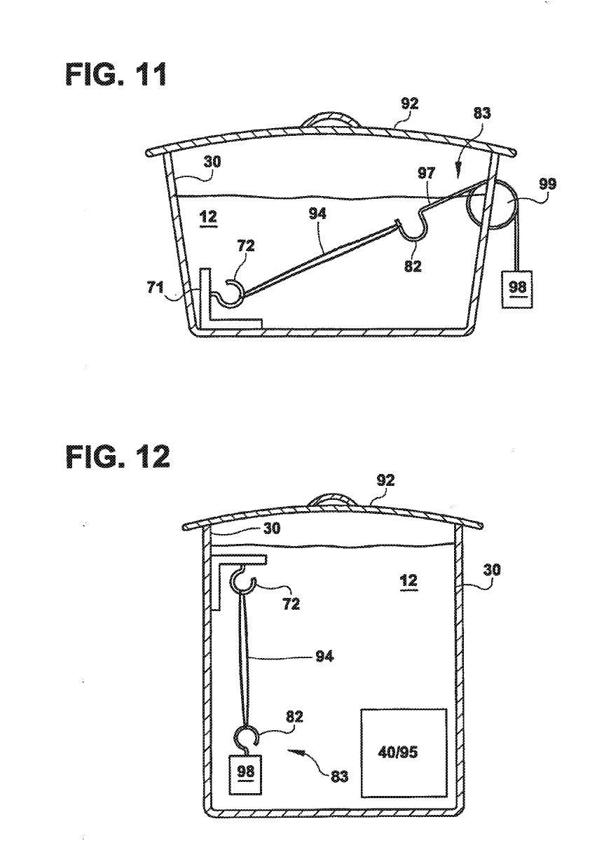

The tray 50 may be of any shape and size appropriate for being disposed within the basin 30. The tray 50 is further designed to account for placement of the heating element 40 within the basin 30. For example, the shape of the tray 50 may be consistent with the shape of the basin 30, as shown in FIG. 2. Alternatively, the tray 50 may be smaller than the floor 34 of the basin 30 as shown in FIG. 3. Further, the tray 50 may comprise a cut away portion 52A as shown in FIG. 1. The tray 50 is preferably made of polyvinylchloride, HDPE, PET, or stainless steel, however any appropriate material is within the inventive concept. The tray 50 preferably comprises perforations 54. The perforations 54 may be circular, as shown in FIG. 1, or may be of any shape, size and number such that fluid may pass through the tray 50. The tray 50 is further designed to receive the graft holding fixture 60 as discussed in detail below. According to one embodiment the tray 50 is suspended above the floor 34 of the basin 30, such that the tray 50 does not rest against the floor 34 of the basin 30, by one or more feet 55 (see FIGS. 4 and 9), a folded edge 56 (see FIG. 5), or the ledge 35 in the wall 33 of the basin 30 (see FIG. 2). According to other embodiments, as shown in FIG. 3, the tray 50 rests against the floor 34 of the basin 30 or is secured to the floor 34 of the basin 30. According to one embodiment, the tray 50 further includes an apparatus 53 to allow for placement of the tray 50 within the basin 30 and removal of the tray 50 from the basin 30. For example, the apparatus 53 may consist of one or more cutaway portions 52B, as shown in FIG. 1. Alternatively, as illustrated in FIGS. 4 and 5, the apparatus 53 may comprise one or more handles 51 secured to the tray 50 and extending above the surface of the fluid 12. According to yet other embodiments, as shown in FIGS. 11 and 12, the device 10 does not include a tray 50.

The graft holding fixture 60 comprises a graft hook 70 and a graft tensioner 80, positioned relative to one another such that a graft 94 may be secured therebetween during preconditioning.

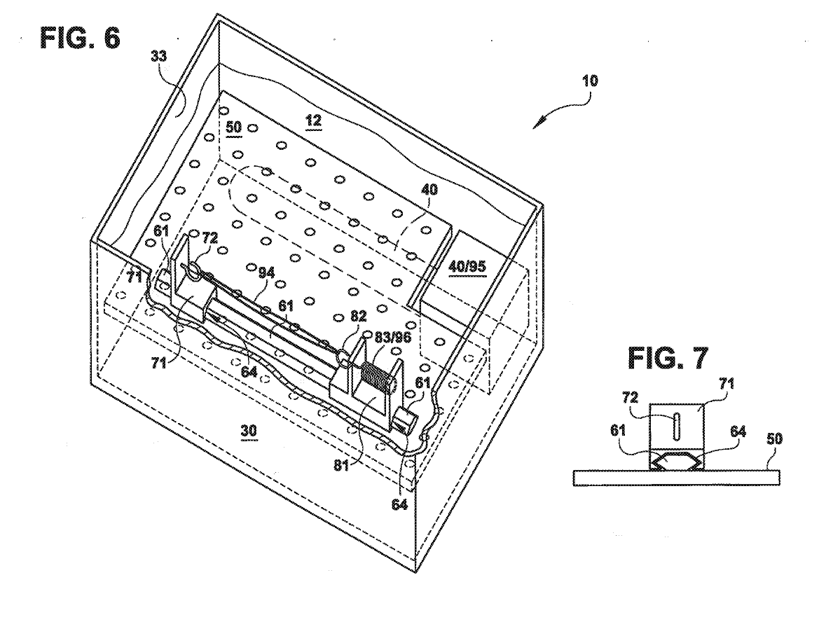

According to one embodiment, both components (70 and 80) of the graft holding fixture 60 are removably secured to the tray 50. According to another embodiment, both components (70 and 80) of the graft holding fixture 60 are permanently secured to the tray 50. According to yet another embodiment, one component (70 or 80) of the graft holding fixture 60 is permanently secured to the tray 50 while the other component (70 or 80) is removably secured to the tray 50 such that the distance between the components 70 and 80 is adjustable. For example, the graft hook 70 and the graft tensioner 80 may be secured to the tray 50 using a peg board system wherein one or more pegs 62 located on the underside of both the graft hook 70 and the graft tensioner 80 are received by one or more perforations 54 of the tray 50. As shown in FIG. 2, a nut-like component 63 may be attached to each peg 62 after the peg 62 is disposed upon the tray 50 in order to secure the graft hook 70 and the graft tensioner 80. According to another embodiment, and as shown in FIGS. 6 and 7, the graft hook 70 and the graft tensioner 80 are mounted upon the rail 61, the rail 61 being removably or permanently secured to the tray 50 by any suitable means. The graft hook 70 and the graft tensioner 80 comprise a recessed portion 64 for receiving the rail 61 and are moveable with respect to the rail 61 such that the distance between the graft hook 70 and the graft tensioner 80 is adjustable. One or more set screws, detents or stops may be used to secure the graft hook 70 and the graft tensioner 80 in a desired position along the rail 61. According to yet other embodiments, the graft hook 70 and/or the graft tensioner 80 may be secured directly to the basin 30.

The graft hook 70, the first element of the graft holding fixture 60, is capable of securing either end, both ends, or a folded portion of a graft 94. According to one embodiment, set forth in FIG. 2, the graft hook 70 includes a base 71, a graft securing element 72 and an attachment mechanism 73. The graft securing element 72 may comprise any means for securing a graft 94, including, but not limited to, one or more hooks, clips, pins, posts, loops or clamps. The attachment mechanism 73 may be the peg 62 affixed to the base 71, the recessed portion 64 for receiving the rail 61, or any other suitable means for permanently or removably attaching the graft hook 70 to the tray 50 or the basin 30. Alternatively, as set forth in FIG. 13, the graft hook 70 includes a weighted bar 101 and the graft securing element 72.

The graft tensioner 80, the second element of the graft holding fixture 60, is capable of securing either end, both ends or a folded portion of a graft 94. According to one embodiment, as set forth in FIG. 2, the graft tensioner 80 includes a base 81, a graft securing element 82, a tensioning component 83, a latching mechanism 84, and an attachment mechanism 85. The graft securing element 82 may comprise any means for securing a graft, including, but not limited to, one or more hooks, clips, pins, posts, loops or clamps. The tensioning component 83 may be any means for applying tension to the graft 94. According to one embodiment, as shown, for example, in FIGS. 1 and 2, the tensioning component 83 is a spring 96 or other linear elastic component. According to that embodiment, the spring 96 may be a weak spring, a strong spring, or anything in between. The elastic properties of the spring 96 should be chosen according to the particular application for which it is intended. For example, a weak spring 96 may prevent slack as the graft 94 relaxes, but may not add significant additional tension to the system. This type of spring 96 may be appropriate for pediatric surgery or a surgery in which a single bundle graft 94 is used. A strong spring 96 may be used to apply significant tension to a graft 94, perhaps during an anterior cruciate ligament reconstruction in which a multi-bundled graft 94 is used. According to one embodiment, the spring 96 is color coded according to its elastic properties, such that a medical professional may easily select the spring 96 that he or she believes is appropriate for a particular application. The latching mechanism 84 is a mechanism for temporarily maintaining the spring 96 in an extended, relaxed or compressed position. The latching mechanism 84 may be used, for example, to maintain the spring 83 in an extended position such that a graft 94 may be readily secured to the graft tensioner 80 by the graft securing element 82. Once the graft 94 is secured to both the graft hook 70 and the graft tensioner 80, the latching mechanism 84 may be released, allowing the spring 83 to retract, thereby applying tension to the graft 94. The attachment mechanism 85 may be the peg 62 affixed to the base 81, a recessed portion 64 for receiving the rail 61, or any other suitable means for permanently or removably attaching the graft tensioner 80 to the tray 50 or the basin 30.

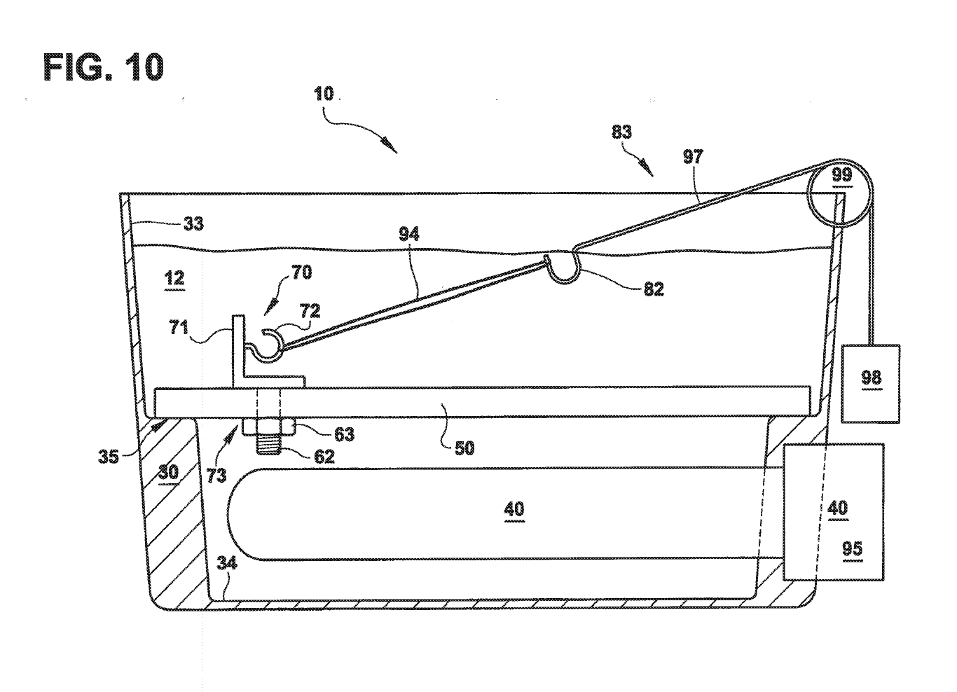

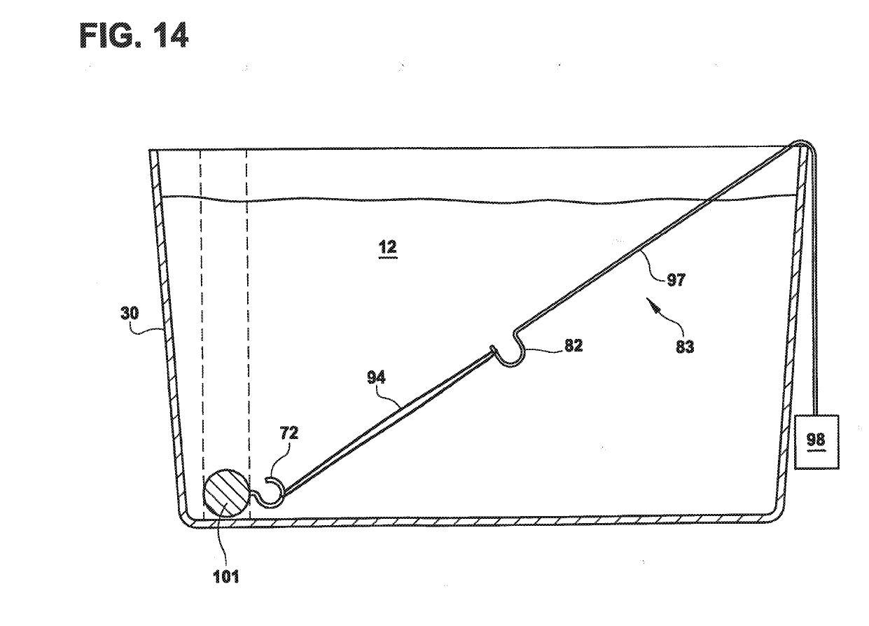

According to another embodiment, as shown in FIGS. 10, 11, 12, 13 and 14, the graft tensioner 80 includes the graft securing element 82 and the tensioning component 83. According to the embodiment set forth in FIGS. 10 and 11, the tensioning component 83 is a gravity based system comprising a string or cable 97 and a weight 98. The weight 98 is attached to the cable 97. The cable 97 is attached to the graft securing element 82 and routed over the wall 33 of the basin 30. According to another embodiment, the graft tensioner 80 may further include a pulley 99 for routing the string or cable 97 over or through the wall 33 of the basin 30. According to yet another embodiment, shown in FIG. 12, the graft 94 may be positioned vertically, for example, by securing the graft hook 70 to the wall 33 of the basin 30. According to this embodiment, the appropriate weight 98 is secured to the graft 94 via the graft securing element 82, and thereby suspended from the graft 94.

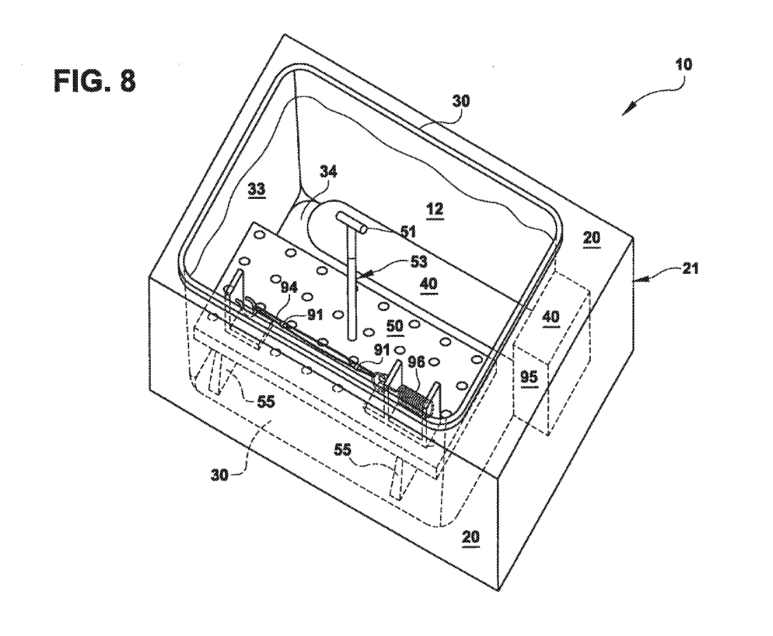

According to yet another embodiment, the graft tensioning device 10 further comprises a base 20. According to this embodiment, shown in FIGS. 8 and 9, the basin 30 and other components of device 10 are disposed within the base 20. The base 20 consists of one or more walls 21 and a floor 22. The base 20 may be any shape or size capable of containing the basin 30 and the fluid 12 disposed within the basin 30. The base 20 is preferably of metal or plastic, however any suitable material is within the inventive concept. According to one embodiment, the base 20 and the basin 30 are two separate and disjoinable component pieces. According to yet another embodiment, the base 20 and the basin 30 are one continuous piece of manufacture. According to either of these embodiments, the heating element 40 is disposed either within the base 20 and within the basin 30, or within the base 20 and exterior to the basin 30.

According to another embodiment, and as shown in FIG. 5, the graft tensioning device 10 further comprises a shelf 90 for supporting the tray 50 outside of the fluid 12. The shelf 90 may be any surface for supporting the tray 50 above the surface of the fluid 12. For example, the shelf 90 may comprise a flat projection extending inward or outward from one side of the base 20 and/or basin 30. Further, the shelf 90 may comprise a lip or ledge for securing the tray 50 above the surface of the fluid 12. According to one embodiment the shelf 90 is permanent. According to other embodiments, the shelf 90 is removable or collapsible. The shelf 90 may support the tray 50 while a medical professional prepares the graft 94 and secures the graft 94 to the graft tensioning fixture 60. The shelf 90 may further be used to support the tray 50 following preconditioning and before implantation.

According to another embodiment, and as shown in FIGS. 8 and 9, the device 10 further comprises one or more tube sizers 91, as known in the art, for placing around the graft 94 to prevent, control and/or monitor the expansion of the graft 94 during preconditioning. According to yet another embodiment, the tube sizer 91 comprises perforation to allow for penetration of the fluid 12. The tube sizer 91 is preferably made of plastic or metal, however any material is within the inventive concept.

Various embodiments of the device further include a cover 92 to assist in maintaining the temperature and/or sterility of the system, among other things.

According to one embodiment, the device 10 is permanent or reusable. For that embodiment, each component part is of a material designed to withstand conditions including moist heat by steam autoclaving, dry heat, ethylene oxide gas and gamma radiation for the purpose of sterilization. According to other embodiments, the device 10 is partially disposable and partially reusable. For example, the base 20 and the heating element 40 may be permanent or reusable, while the basin 30, the tray 50 and the graft holding fixture 60 may be disposable. According to yet another embodiment, the device 10 is disposable.

The graft tensioning device 10 is suitable for use within an operating room. While in use, the basin 30 contains an appropriate fluid 12. The graft hook 70 and the graft tensioner 80 are secured to the tray 50 by any suitable means. According to one embodiment, the tray 50 may rest on shelf 90 during the preparation of the device and the harvesting of the graft 94. According to one embodiment, a medical professional selects the linear elastic element 96 appropriate for a particular application. The linear elastic element 96 is secured to the graft tensioner 80. The linear elastic element 96 may be placed in an extended position and temporarily maintained in the extended position by the latching mechanism 84. Once the graft 94 is harvested from a patient or otherwise obtained, the graft 94 is secured to the graft hook 70, by graft securing element 72, and the graft tensioner 80, by graft securing element 82. Once the graft is appropriately secured, the latching mechanism 84 may be released, thereby applying tension to the graft 94. According to another embodiment, the medical professional selects the weight 98 appropriate for a given application. The weight 98 is secured to the graft 94 by either the cable 97 and/or the graft securing element 82. According to any of the foregoing embodiments, a tuber sizer 91 may be positioned around the graft prior to securing the graft to the graft hook 70 and the graft tensioner 80. Once the graft 94 is affixed to the graft holding fixture 60, the medical professional may use apparatus 53 to lower the tray 50 into the basin 30 containing fluid 12. The time, temperature and tension necessary to condition the graft is determined by an appropriate medical professional. Prior to implantation of the graft 94, the tray 50 is removed from basin 30 using apparatus 53.

While the invention has been described with reference to a preferred embodiment or embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to a particular embodiment disclosed as the best mode contemplated for carrying out this invention, but that the invention will include all embodiments falling within the scope of the appended claims.

What is claimed is:

1. A method of conditioning a tissue graft prior to implantation, comprising:
using a basin filled with warm fluid;
securing a tissue graft to a graft hook within the basin; and
securing the tissue graft to a graft tensioner within the basin, and
wherein after securing the tissue graft to the graft hook and to the graft tensioner, the tissue graft is submerged within the warm fluid of the basin.

2. The method of claim 1, wherein the graft tensioner comprises a linear elastic element that applies tension to the tissue graft.

3. The method of claim 1, further comprising securing a weight to the graft tensioner such that the weight applies tension to the tissue graft.

4. The method of claim 1, further comprising placing a tube sizer around the tissue graft before submerging the tissue graft in the warm fluid, wherein the tube sizer controls expansion of the graft.

5. The method of claim 1, further comprising heating fluid within the basin to produce the warm fluid.

6. The method of claim 5, wherein heating fluid within the basin comprises activating a heating element that is secured within the basin to heat fluid within the basin.

7. The method of claim 6, wherein the heating element is permanently secured within the basin.

8. The method of claim 6, wherein the heating element heats the fluid within the basin to about body temperature.

9. The method of claim 6, further comprising removably securing the graft hook and the graft tensioner to a tray, wherein the tray is selectively removable from the basin.

10. The method of claim 9, wherein the tray defines a plurality of perforations, and wherein removably securing the graft hook and the graft tensioner to the tray comprises selectively engaging at least one perforation of the tray with at least one of the graft hook and the graft tensioner.

11. The method of claim 9, wherein each of the graft hook and the graft tensioner further comprises a base, a graft securing element, and an attachment mechanism, and wherein removably securing the graft hook and the graft tensioner to the tray comprises using the attachment mechanism to secure the respective graft hook and graft tensioner to the tray.

12. The method of claim 1, further comprising adjusting a distance between the graft hook and the graft tensioner to account for graft size variations.

13. The method of claim 1, wherein the tissue graft is a bone-tendon-bone graft.

14. The method of claim 1, wherein the warm fluid is a sterile saline solution.

15. The method of claim 1, wherein the warm fluid is an antibiotic rinse solution.

16. The method of claim 2, further comprising using a latching mechanism to maintain the linear elastic element in an extended position before the tissue graft is secured to the graft tensioner.

17. The method of claim 16, further comprising releasing the latching mechanism when the tissue graft is secured to the graft tensioner, thereby permitting application of tension to the tissue graft by the graft tensioner.

18. The method of claim 9, further comprising, before the graft hook and the graft tensioner are secured to the tray, positioning the tray in an elevated position relative to the fluid within the basin, wherein the graft hook and the graft tensioner are secured to the tray when the tray is in the elevated position, and wherein the tissue graft is secured to the graft hook and to the graft tensioner when the tray is in the elevated position.

19. The method of claim 18, wherein positioning the tray in the elevated position comprises resting the tray on a shelf defined by the tray.

20. The method of claim 18, further comprising lowering the tray into the fluid within the basin.
